(19) United States
(12) Patent Application Publication
(10) Pub. No.: US 2009/0012410 A1
(43) Pub. Date: Jan. 8, 2009

(54) Blood Pressure Monitor and Method for Operating Same

(75) Inventors: Uwe Diegel, Paris (FR); Gerhard Frick, Feldkirch (AT)

Correspondence Address: SHOEMAKER AND MATTARE, LTD, 10 POST OFFICE ROAD - SUITE 110, SILVER SPRING, MD, 20910, US

(73) Assignee: MICROLIFE INTELLECTUAL PROPERTY GMBH, Widnau (CH)

(21) Appl. No.: 11/577,022

(22) PCT Filed: Oct. 6, 2005
(86) PCT No.: PCT/EP05/55063

§ 371 Date: Apr. 11, 2007

(30) Foreign Application Priority Data
Oct. 11, 2004 (EP) 04024158.0

(51) Int. Cl. A61B 5/022

(52) U.S. Cl. 600/490

(57) ABSTRACT

A blood pressure measuring device for measuring the blood pressure of a patient is provided. The device comprises an inflatable cuff adapted to be placed around the upper arm or wrist of the patient, a pressure sensor for measuring the pressure within the cuff, a calculating unit for oscillometrically calculating the value of the blood pressure on the basis of the pressure values in the cuff, means for inflating the cuff and pressure control means for controlling and releasing the pressure in the cuff, said pressure control means comprising at least one valve. Further, the device has switching means for switching between at least two measuring modes, wherein in a first measuring mode, the cuff pressure is released at a first deflation rate and in a second measuring mode the cuff pressure is released at a second deflation rate which is smaller than the first deflation rate.

The invention relates to a blood pressure monitor and a method for operating a blood pressure monitor according to the preamble of the independent patent claims.

It is known to measure the blood pressure of a patient by applying an inflatable cuff around the upper arm of the patient. The pressure values within the cuff are conventionally displayed with a mechanic or with a mercury manometer. A stethoscope is placed between the upper arm of the patient and the inflated cuff. The pressure values displayed on the manometer, when Korotkoff sounds appear or disappear during deflation of the cuff, give the systolic and diastolic blood pressure values. This blood pressuring method is appreciated by doctors because of the sufficiently high accuracy achieved through simple devices and because of the possibility of additional diagnostics when using a stethoscope. In the following, this method for measuring the blood pressure will be referred to as acoustic method.

It is further known to measure the blood pressure completely electronically, e.g. based on an oscillometric method. Depending on a pressure signal measured within the cuff, the values of the systolic and the diastolic blood pressure are calculated with an algorithm and displayed on a digital display. In the following, such a method will be referred to as automatic method.

In EP 1 272 103 a blood pressure measurement system is presented, where the blood pressure is determined on the basis of different methods. One method is to determine the blood pressure on the basis of Korotkoff sound, using a microphone inside the cuff. Another method is the oscillometric method. The measure-ments by different methods are performed automatically and simultaneously. A blood pressure value is displayed which is calculated with respect to all the different measurement results.

From EP 422 512 an electronic sphygmomanometer is known, where the blood pressure is measured as well as using the Korotkoff method and the oscillometric method. The Korotkoff method is performed automatically with the help of a microphone. The blood pressure is displayed on the basis of either measurement.

With both these devices it is possible to measure the blood pressure on the basis of different measurement methods. But with none of these devices it is possible to perform an acoustic measurement in a conventional manner, i.e. with a stethoscope. Still, this method is preferred by many physicians.

It is an object of the present invention to overcome the drawbacks of the prior art, especially to provide a device with which it is possible to measure the blood pressure automatically and acoustically.

This object is solved by a device for measuring the blood pressure of a patient and a method for operating a blood pressure monitor according to the independent patent claims.

The blood pressure measuring device comprises an inflatable cuff which is adapted to be placed around the upper arm or wrist of the patient. It is also conceivable that the cuff is adapted for the use on other body parts, e.g. the leg of a patient.

Further, the device comprises a pressure sensor for measuring the pressure within the cuff and a calculating unit for automatically, in particular oscillometrically, calculating the value of the blood pressure on the basis of the pressure values in the cuff.

The cuff is inflated by means for inflating the cuff. This can be an electric pump, but it is also possible that the cuff is inflated manually with the help of an inflation bulb.

The device further comprises pressure control means for controlling and releasing the pressure in the cuff. Said pressure control means comprise at least one valve.

The device has switching means for switching between at least two measuring modes, wherein in a first measuring mode, the cuff pressure is released at a first deflation rate and in a second measuring mode the cuff pressure is released at a second deflation rate which is smaller than the first deflation rate. It is also possible that the switching means allow a continuous regulation of the deflation rate between a higher and a lower limit.

It is preferred, that at least in the second measuring mode the current cuff pressure is indicated on a display during the deflation, such that an acoustic blood pressure measurement based on Korotkoff sounds is feasible. Alternatively it would be possible to have a switch to turn the display on and off if required, irrespective of the mode in which the device is used.

The device according to the present invention combines the advantages of the automatic, e.g. oscillometric measurement method with the advantages of acoustic measurement method. Many physicians prefer the acoustic method. With a device according to the present invention, they can control the automatic measurement and themselves by measuring the blood pressure acoustically and automatically at the same time. Further, the device according to the present invention is very flexibly applicable. To increase the comfort for the patient, an automatic measurement with a higher deflation rate can be performed. Thus, the measurement is faster and the patient does not have to wait that long. To verify the accuracy of the measurement result, both methods can be applied at the same time. To save energy, the oscillometric measurement can be turned off.

In a special embodiment of the invention, the device is provided with a switch to optionally turn off the automatic measurement in the second measuring mode. Then, only an acoustic measurement can be performed in the second measuring mode.

In a further embodiment, the pressure control means are switchable between three pressure release modes, where in the third mode the pressure is released quickly after a performed measurement. This third mode can be activated manually. Alternatively, the third mode can be automatically initiated when enough data has been collected to calculate the blood pressure, or when the cuff pressure reaches a predetermined threshold value.

This embodiment has the advantage that the comfort for the patient is increased. It is uncomfortable for the patient to measure the blood pressure because of the pressure of the cuff. Therefore, it is very convenient to deflate the cuff as soon as possible at a maximum deflation rate.

It is possible, that the pressure control means comprise at least two different valves. According to the position of the switching means, the cuff is deflated through either of these valves. Alternatively, the pressure control means comprise at least one valve with different deflation rates. For instance, this might be voltage driven valves where the deflation rate directly depends on the drive voltage. Preferably, the valve with different deflation rates is electromagnetically activatable. With such a valve, even a continuous regulation of the deflation rate is feasible. This results in an ?infinite’ number of measuring modes, as every deflation rate defines a measuring mode.

In one particular embodiment of the invention, the blood pressure measuring device is a hand held device with a bulb for manually inflating the cuff. Such a device is known from EP 1 333 752. The advantage of such a device is, that the energy consumption of the device is reduced strongly by manually inflating the cuff. This makes the measurement cheaper, as the battery of the device does not need to be replaced that often. Further, the device is lighter, as no pressure pump and smaller batteries are used. Therefore, such a device is particularly suitable for the use in isolated locations.

To save energy, the device can be provided with an on/off switch. If the cuff is inflated manually with a bulb, the switch can be activatable through a first compression of the bulb. Otherwise, conventional on/off buttons are used.

It is particularly user-friendly to design the device such as to indicate when the pressure in the cuff has reached a setup value or such as to indicate when the pressure in the cuff has not yet reached a setup value. This feature provides an additional security to the user that the measurement result is reliably accurate. Preferably, the setup value is determined by the calculating unit during the inflation of the cuff on the basis of a rough estimation of the systolic blood pressure of the patient. The setup value should be set about 20 to 30 mmHg above the systolic blood pressure to allow for an accurate measurement.

Additionally or alternatively, the calculating unit could have a memory unit where the setup values of a number of patients could be stored. This is suitable, when one device is used only for a small number of patients. The setup value would then be preselected from the memory.

The memory could also be designed to store the setup value of the last measurement. This can be useful, when two subsequent measurements are performed on one patient.

Alternatively, the setup value could be a preset value which is independent from the patient. This value could be set e.g. at 160 mmHg. For patients with a low blood pressure the setup value might be, however, too high, and the discomfort of the cuff is increased. For people with a high blood pressure the preset setup value can bee to low, such that it is necessary to inflate the cuff additionally.

In a preferred embodiment it is checked if the setup value is high enough before the cuff pressure is released. If required, the cuff can be inflated additionally before releasing the pressure.

The method for operating a blood pressure measuring device comprising an inflatable cuff, pressure control means for controlling and releasing the pressure in the cuff and switching means to switch between at least two measuring modes, comprises the steps of

starting a measurement,

checking, whether the method is to be done in a first or in a second measuring mode,

inflating the cuff,

carrying out a measurement,

wherein in a first measuring mode the cuff pressure is released at a first deflation rate, and the blood pressure is determined automatically,

and in a second measuring mode the cuff pressure is released at a second deflation rate which is smaller than the first deflation rate, and the current cuff pressure is indicated on a display.

The steps are not mandatory to be carried out in the indicated order. For example, it is possible to inflate the cuff first and to subsequently check, whether the method is to be done in a first or in a second measuring mode.

In the second measuring mode the current cuff pressure has to indicated on the display. However, it is not excluded that the current cuff pressure is indicated in the first measurement mode as well. For someone using the blood pressure monitor it might be interesting to see the current cuff pressure in any case.

As well, it is possible to automatically determine the blood pressure in the second measuring. This has the advantage, that the blood pressure can be determined in two ways with one single measurement. On the other hand, if no automatic measurement is performed in the second measuring mode, the energy consumption of the device is reduced. The invention will be more clearly understood with the aid of the following description of some embodiments of the invention and the accompanying drawings which show:

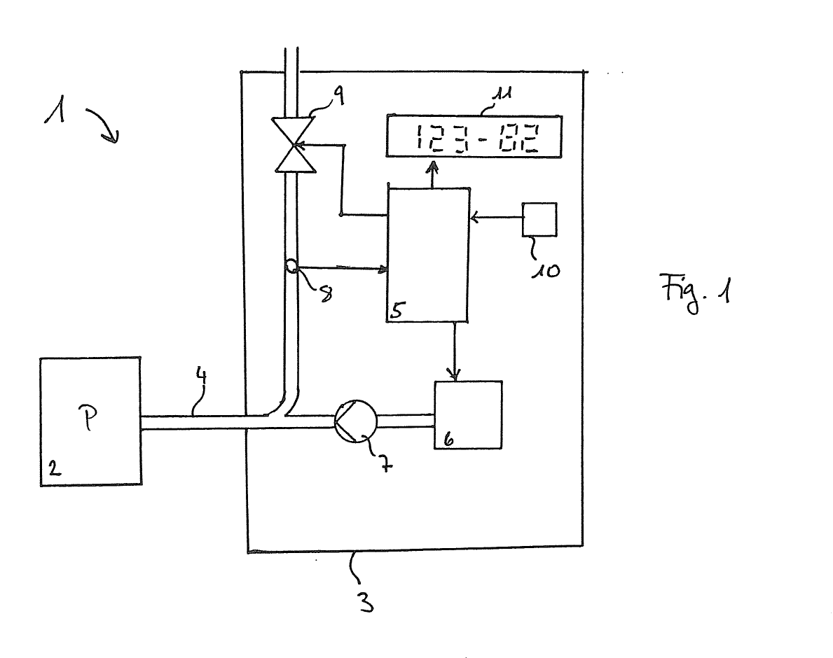

FIG. 1: a schematic view of a device according to the present invention,

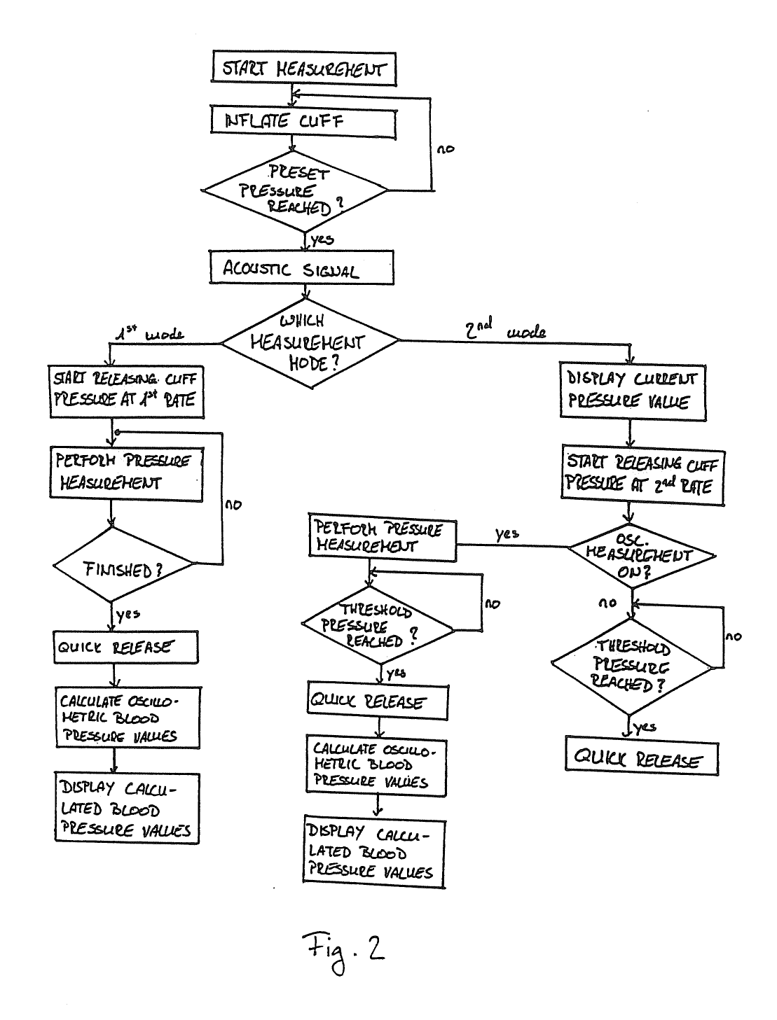

FIG. 2: a flow chart of the method according to the present invention, and

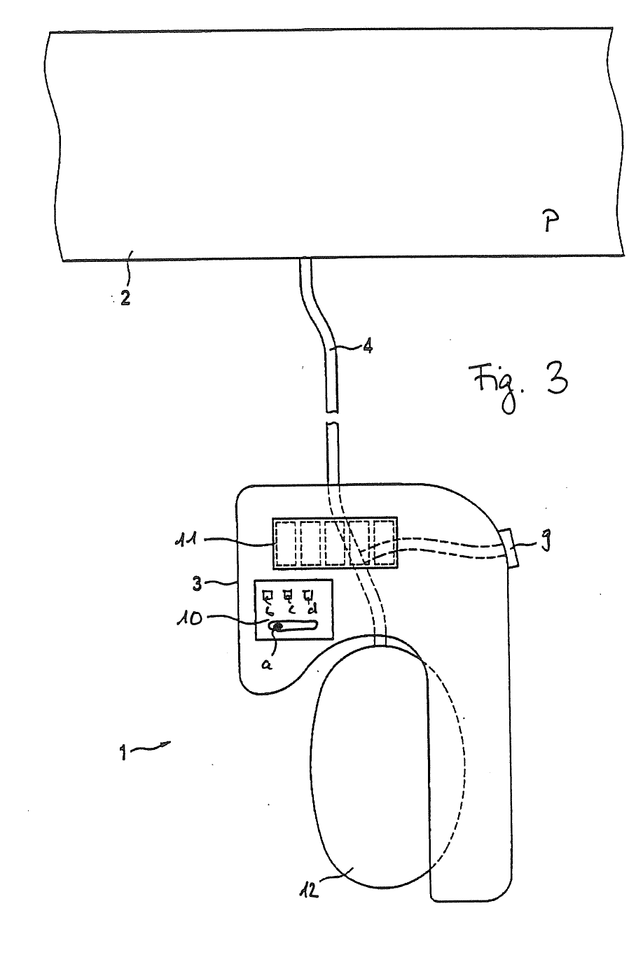

FIG. 3: an embodiment of a device according to the present invention.

The device 1 according to FIG. 1 comprises a cuff 2 and a housing 3. The cuff 2 is connected to the housing 3 via tube 4 which is usually a flexible rubber tube. The cuff is inflated by a pressure pump 6 through an inflation check valve 7. The cuff pressure P is measured with a pressure sensor 8.

The operation of the device 1 is controlled by a calculating unit 5. This calculating unit 5 gets inputs from internal and external sources and processes this information.

When a measurement is started, the cuff 2 is inflated by the pressure pump 6. The pressure pump is controlled by the calculating unit 5 on the basis of the pressure value P derived by the pressure sensor 8. When a setup pressure value is reached, the pressure pump 6 is stopped, and a measurement can be performed. The setup value is determined during the inflating of the cuff on the basis of a rough estimation of the systolic blood pressure. The setup value is set at 30 mmHg above the estimated systolic blood pressure. For the measurement, the cuff 2 is deflated by means of deflation means 9 that are controlled by the calculating unit 5 as well.

By a switch 10 the user can choose between two measurement modes. In the first measurement mode, the cuff pressure P is released more quickly than in the second mode. Thus, in the second mode an acoustic measurement is feasible, whereas in the first mode an oscillometric measurement is performed at minimum time. At the second measurement mode, the current cuff pressure P is displayed on a LED-display 11.

The calculating unit 5 receives information about the selected mode from the position of switch 10. In dependency of the switch position, the cuff pressure P is released faster or slower through deflation means 9. The deflation means 9 may comprise several valves, but in this embodiment only one adjustable valve 9 is used.

The deflation valve 9 is an electrically driven valve of the type VS-LMOl of SUMMIT ELEC-TECH CO., LTD. The deflation rate of the valve is adjusted by a drive voltage. The voltage applied to valve 9 is controlled by the calculating unit 5 in dependency of the position of switch 10. Thus it is possible to deflate the cuff 2 at different deflation rates with valve 9 only.

In the first measurement mode, data is collected until there is enough data to calculate the systolic and diastolic blood pressure values in the calculating unit 5. Once there is enough data, the cuff pressure P is released completely as fast as possible. Meanwhile, the blood pressure values are calculated in the calculating unit 5. Finally, the calculated values are displayed on the display 11. FIG. 2 is a flow chart of an embodiment of the method according to the present invention. Before a measurement is started, one of two possible measurement modes is selected by the user. Then, the user starts a measurement with an on-command.

When a measurement is started, the cuff 2 is inflated until a setup pressure value is reached. The setup value is determined during the inflation of the cuff on the basis of a rough estimation of the systolic blood pressure. The setup value is set at 30 mmHg above the estimated systolic blood pressure.

The current cuff pressure P is observed by the calculating unit 5. Pressure pump 6 is controlled and operated by the calculating unit 5 in dependency of the cuff pressure P. As soon as the setup pressure is reached, an acoustic signal is issued to inform the user. Then it is checked by the calculating unit 5, which measuring mode is selected.

If the first measuring mode is selected, the cuff pressure P is released at the first deflation rate. This rate is that fast, that an oscillometric measurement can be performed. The first deflation rate is usually about 4 to 6 mmHg/sec between the systolic and diastolic blood pressure. Oscillometric data is collected until the calculating unit 5 receives enough data to calculate the blood pressure values. As soon as the calculating unit has enough data, the cuff pressure P is released quickly against zero in about 3 to 8 seconds. The pressure release time is depending on the remaining pressure which is further on depending on the patients systolic blood pressure.

Meanwhile, the systolic and diastolic blood pressure values are calculated by the calculating unit 5. When the calculation is finished, the calculated blood pressure values are indicated on the display 11.

If the second measuring mode is selected, the current cuff pressure P is displayed on the display 11. The pressure P is released at a second deflation rate. The second deflation rate is smaller than the first deflation rate, it is about 2 to 3 mmHg/sec. Thus, it is slow enough, that the user can perform an acoustic blood pressure measurement with the stethoscope. The user decides by a switch 10d (FIG. 3), if additionally an oscil-lometric measurement should be performed.

If it is switched on, the oscillometric measurement is carried out analogue to the first measuring mode. The cuff pressure P is released at the second deflation rate until a threshold pressure value is reached. This threshold value can be e.g. 50 mmHg, depending on the patient's oscillometric determined diastolic pressure. It is important that the threshold value is below the patient's oscillometric diastolic blood pressure value, such that the acoustic diastolic blood pressure of the patient can be determined reliably. When the threshold pressure is reached, the cuff pressure P is released quickly to conclude the measurement as soon as possible. Meanwhile, the blood pressure values are calculated by the calculating unit 5 and displayed on the display 11.

If the oscillometric measurement is switched off, the cuff pressure P is released at the second deflation rate, but no calculation is performed. When a threshold pressure is reached, the cuff pressure P is quickly released. The threshold pressure depends on the patient and can be chosen from a memory by the user. As no automatic measurement was performed, no measurement results are displayed. Optionally, the display is turned off automatically, when the cuff pressure P is released completely. Alternatively, Mone’ or any similar expression could be written on the display.

A preferred embodiment of the device according to the present invention is illustrated with reference to FIG. 3. The device 1 comprises a hand held housing 3 and a cuff 2. The cuff is manually inflated by the inflation bulb 12. Thus, the energy consumption of the device 1 is smaller than with a conventional blood pressure monitor. Further, as in this embodiment no pressure pump 5 is needed, the device 1 is not as heavy and is thus very suitable for the use in remote locations.

The cuff 2 is connected to the housing 3 and the inflation bulb 12 by a flexible tube 4. The switching means 10 comprise switches to select the mode (a), to start a measurement (b), to turn the display on or off (c), and to turn the oscillometric measurement on or off (d).

Further, the device comprises a LED-display 11 and a electrically driven valve 9. The valve 9 corresponds to the valve 9 of FIG. 1.

The calculating unit is turned on by the first compression of the bulb 12 and is automatically turned off 60 seconds after the complete deflation of the cuff 2. This time interval can be adjusted manually according to the preferences of the user.

1. A blood pressure measuring device for measuring the blood pressure of a patient, comprising
an inflatable cuff,
a pressure sensor for measuring the pressure within the cuff,
a calculating unit for automatically, preferably oscillometrically, calculating at least one value of the blood pressure on the basis of the pressure values in the cuff,
means for inflating the cuff,
and pressure control means for controlling and releasing the pressure in the cuff,
wherein the device has switching means for switching between at least two measuring modes, wherein in a first measuring mode, the cuff pressure is releasable at a first deflation rate and in a second measuring mode the cuff pressure is releasable at a second deflation rate which is smaller than the first deflation rate.

2. A blood pressure measuring device according to claim 1, wherein at least in the second measuring mode the current cuff pressure is indicated on a display during the deflation, such that an acoustic blood pressure measurement based on Korotkoff sounds is feasible.

3. A blood pressure measuring device according to claim 2, wherein the device is provided with a switch to optionally turn off the oscillometric measurement in the second measuring mode.

4. A blood pressure measuring device according to claim 1, wherein the pressure control means are switchable between three pressure release modes, wherein in the third mode the cuff pressure is released faster than in the first measuring mode.

5. A blood pressure measuring device according to claim 1, wherein the pressure control means comprise at least two different valves.

6. A blood pressure measuring device according to claim 1, wherein the pressure control means comprise at least one valve which is operatable with different deflation rates.

7. A blood pressure measuring device according to claim 6, wherein the valve with different deflation rates is electromagnetically activatable.

8. A blood pressure measuring device according to claim 1, wherein the blood pressure measuring device is a hand held device with a bulb for manually inflating the cuff.

9. A blood pressure measuring device according to claim 1, wherein the device is provided with an on/off switch.

10-11. (canceled)

12. A device according to claim 9, when the switch is activatable through a first compression of the bulb.

13. A blood pressure measuring device according to claim 1, wherein the device is designed such as to indicate when the pressure in the cuff has reached a setup value or such as to indicate when the pressure in the cuff has not yet reached a setup value.

14. A method for operating a blood pressure measuring device comprising an inflatable cuff, pressure control means for controlling and releasing the pressure in the cuff and switching means to switch between at least two measuring modes, comprising the steps of
starting a measurement,
checking, whether the method is to be done in a first or in a second measuring mode,
inflating the cuff,
carrying out a measurement,
wherein in a first measuring mode the cuff pressure is released at a first deflation rate, and the blood pressure is determined automatically,
and in a second measuring mode the cuff pressure is released at a second deflation rate which is smaller than the first deflation rate, and the current cuff pressure is indicated on a display.
